(19) United States
(12) Patent Application Publication
(10) Pub. No.: US 2008/0274182 A1
(43) Pub. Date: Nov. 6, 2008

(54) Tablet coatings made from modified carboxymethylcellulose materials

(76) Inventors: Regina Helena Alida Boekema, Deventer (NL); Henrica Wilhelmina Cornelia Vaessen-van Hoven, Voorthuizen (NL); Anja Maria Christina Petronella, Beuningen (NL)

Correspondence Address: J.M. Huber Corporation;Legal Department, 333 Thornall Street, Edison, NJ, 08837, US

(21) Appl. No.: 11/799,991

(22) Filed: May 3, 2007

(51) Int. Cl. A61K 9/36; A61K 31/717; A61K 9/14

(52) U.S. Cl. 424/480; 424/494; 514/781

(57) ABSTRACT

Coated tablets for the delivery of active ingredients to a user are provided. Such tablets include particular molecular weight-modified carboxymethylcellulose (CMC) coating materials either alone or in combination with other types of hydrocolloids, biogums, cellulose ethers, and the like. The utilization of such modified CMC products aids in the production of such coatings through the availability of larger amounts of base materials with lower amounts of water requiring evaporation therefrom. In such a manner, not only may dimensionally stable, non-tacky, salt tolerant, and quick dissolving edible coatings be produced, but the amount of time required for such manufacture is minimal when compared with traditional methods of production with -based materials. Furthermore, such novel edible non-digestible tablet coatings exhibit delayed dissolution beyond a user's oral cavity for tastemasking purposes, as well as protection of the tablet from environmental conditions and low tackiness properties to prevent adhesion to the user's palate. The novel method of tablet coating manufacture as well as the ultimate coated tablets exhibiting such physical characteristics are also encompassed within this invention.

### FIELD OF THE INVENTION

This invention relates to coated tablets for the delivery of active ingredients to a user. Such tablets include particular molecular weight-modified carboxymethylcellulose (CMC) coating materials either alone or in combination with other types of hydrocolloids, biogums, cellulose ethers, and the like. The utilization of such modified CMC products aids in the production of such coatings through the availability of larger amounts of solids with lower amounts of water requiring evaporation therefrom. In such a manner, not only may dimensionally stable, non-tacky, salt tolerant, and quick dissolving edible coatings be produced, but the amount of time required for such manufacture is minimal when compared with traditional methods of production with cellulose -based materials. Furthermore, such novel edible non-digestible tablet coatings exhibit increased strength, delayed dissolution beyond a user's oral cavity for tastemasking purposes, as well as protection of the tablet from environmental conditions and low tackiness properties to prevent adhesion to the user's palate. The novel method of tablet coating manufacture as well as the ultimate coated tablets exhibiting such physical characteristics are also encompassed within this invention.

### BACKGROUND OF THE INVENTION

Coated tablets exhibit the ability to prevent tasting of the tablet filler and/or active until it passes through the user's oral cavity. Additionally, in order to permit ease in swallowing, such coatings prevent adhesion of the tablet to inner mouth surfaces. Also, aesthetic properties, in terms of clear coatings, printed coatings, and low friability tablets, are also possible through the utilization of such coatings. Furthermore, such a coating provides a layer of protection for the active component therein from environmental exposure as well as from crushing during storage and manufacture, as well as increased strength of tablets. Lastly, such a coating provide a longer duration of pharmacological response after the administration of the dosage form than is ordinarily experienced after the administration of an immediate release dosage form. Such extended periods of response provides for many inherent therapeutic benefits that are not achieved with short acting, immediate release products. In essence, the stability of a pharmaceutical dosage form is related to maintaining its physical, chemical, microbiological, therapeutic, pharmaceutical, and toxicological properties when stored, i.e., in a particular container and environment.

Hydrophobic polymers such as certain alkyl cellulose derivatives, zein, acrylic resins, waxes, higher aliphatic alcohols, and polylactic and polyglycolic acids have been used in the prior art to develop tablet coatings. Methods of using these polymers to develop coated tablets involve coating the individual dosage units with these hydrophobic polymers. It is known in the prior art that these hydrophobic coatings can be applied either from a solution or suspension. Since most of these polymers have a low solubility in water, they are usually applied by dissolving the polymer in an organic solvent and spraying the solution onto the individual drug forms (such as beads or tablets) and evaporating off the solvent.

Aqueous dispersions of hydrophobic polymers have been used in the prior art to coat pharmaceutical tablet forms for aesthetic reasons. However, these dosage forms are used for immediate release administration of the active drug contained in the dosage form.

The ingredients used in such tablet coating formulations often present special problems with regard to their physical stability during storage. For example, waxes which have been used in such formulations are known to undergo physical alterations on prolonged standing, thus precautions are taken to stabilize them at the time of manufacture or to prevent the change from occurring. Fats and waxy materials when used in purified states are known to crystallize in unstable forms, causing unpredictable variations in availability rates during stability testing at the time of manufacture and during later storage. Sugars have also been used for coating purposes and can provide taste improvements. However, such components are also undesirable for tackiness problems and caloric intake increases, not to mention certain complexity problems as well.

It is known that certain strategies can be undertaken to obtain stabilized pharmaceutical active formulations in many cases, such as insuring that the individual ingredients are in a stable form before they are incorporated into the product, and that processing does not change this condition, delaying the instability by including additional additives, and inducing the individual ingredients of the dosage form to reach a stable state before the product is finally completed. This adds complexity and cost to tablet production methods, however.

It is also recognized that the moisture content of the pharmaceutical active and filler components can also influence the stability of the tablet. Changes in the porosity and/or hydration level of a polymeric film, such as the ethyl celluloses, can alter the rate of water permeation and drug availability from within a coated tablet. Also, binders such as acacia are known to become less soluble when exposed to moisture and heat. Such problems have been handled by controls in the processing method and proper packaging of the product. Again, however, these methods are quite complex and ultimately expensive to follow.

Furthermore, the use of organic solvents in the preparation of polymer tablet coatings is considered problematic as the formulations have inherent problems with regard to flammability, carcinogenicity, and safety in general. In addition, the use of organic solvents is disfavored due to environmental concerns.

Therefore, it is desirable to prepare a coated tablet prepared from an aqueous solution of a hydrophilic polymer that does not require organic solvent or water in relatively large amounts, thereby permitting ease in evaporation while still yielding an effective, protective, non-tacky, additive-compatible coating applied thereto. However, to date, attempts to prepare such coated tablets using aqueous solutions of hydrophilic polymers have been unsuccessful due to stability problems and such excess drying and/or evaporation times required therefore.

### ADVANTAGES AND SUMMARY OF THE INVENTION

It is therefore an advantage of the present invention to provide a new coating for tablet formulations that does not require excessive drying times or high-energy output drying methods to effectively apply such coating to a tablet surface. Another advantage of this invention is the ability of such a coated tablet to perform at the same level of effectiveness of other coated tablets in various manners.

The present invention encompasses a tablet coated with a composition comprising modified CMC materials exhibiting a molecular weight range of from 1500 to 75000 and a degree of substitution of less than about 1.5; wherein said composition optionally comprises one polymeric additive other than said modified CMC materials. Also encompassed is a method of producing such a tablet coating comprising the steps of a) providing a CMC material exhibiting a molecular weight range of from 80000 to 3000000 and degree of substitution of less than about 1.5; b) degrading said CMC materials by exposing said materials to an enzyme in an amount and for a period of time sufficient to reduce the molecular weight range of said CMC materials to a range of from 1500 to 75000; c) inactivating said enzyme; d) producing a solution of the resultant modified CMC materials of step “b” with at most 90% by weight of water and optionally including at most 12.5% of a plasticizer; e) providing a solid tablet formulation; and f) applying said resultant modified CMC materials of step “d” to the at least a portion of the surface of said tablet formulation of step “e”, thereby allowing said water therein to evaporate therefrom. Such tablet coatings thus exhibit at least the same film strength, delayed dissolution, and active protection capabilities as previously made tablet coatings, but with lower manufacturing costs, and potentially reduced stickiness to the palate, increased ease in swallowing as those currently utilized within the pertinent markets. Such an improvement has been realized through the utilization of a single modified CMC component, thereby permitting a reduction in manufacturing complexity of films. Such is a significant benefit over the comparative prior coating compositions that have relied upon combinations of ingredient polymers to provide similarly effective tablet coatings. Although a single modified CMC polymer may be utilized for this application, it is noted that combinations of the required modified CMC polymer with other polymeric additives, such as hydrocolloids, biogums, sugars, and cellulose ethers may be practiced as well. Such a tablet coating of the modified CMC alone or in combination with such other optional gel-forming or non-gelling viscosity building additives is thus highly desired from a cost perspective as well as effectively delayed dissolution when exposed to the moist environment within a user's oral cavity. Such a specific characteristic is advantageous since quickly dissolved coatings may impart undesirable taste of the tablet formulation (including an active, and fillers, such as various types of salts) to the user.

### DETAILED DESCRIPTION OF THE INVENTION

The aqueous solutions of hydrophilic polymers used as coatings in the present invention may be used in conjunction with tablets, spheroids (or beads), microspheres, seeds, pellets, ion-exchange resin beads, and other multi-particulate systems in order to obtain a desired controlled release of the therapeutically active agent.

The coating formulations of the present invention should be capable of producing a strong, continuous film that is smooth and elegant, capable of supporting pigments and other coating additives, non-toxic, inert, and tack-free.

Such edible coatings are generally comprised of non-toxic ingredients that permit such desirable properties and can easily be applied to tablets of different shapes and sizes. Gelatin has traditionally been the material of choice within the tablet coating (as well as orally ingested capsule) industry. Gelatin exhibits a number of properties that make such a material a proper candidate for tablet coating including good film forming properties (strength and flexibility, primarily), good solubility in biological fluids at typical body temperature, low viscosity at 50° C. at high solids concentrations, and a gel state at low temperatures. Likewise, ethylcellulose and methylhydroxypropyl cellulose have recently found favor within the tablet industry for the same basic reasons.

Other typical coatings comprise polymers and films such as pullulan, cellulosics (such as hydroxypropyl cellulose and carboxymethyl cellulose) and sugars, carrageenan, pectin, as well as mixtures of certain low molecular weight varieties of products and high molecular weight types. Although such coatings have been produced in large-scale methods over the last few years, there are certain limitations that are either aesthetically questionable to the consumer or include increased manufacturing costs that are passed on from the tablet manufacturer to the consumer ultimately.

As noted above, such previously used polymers exhibit certain drawbacks, unfortunately, and particularly in terms of costs of manufacture and application to target tablets. As noted above, high clarity and low tackiness are generally properties sought after by the consumer. Clear, transparent films give an appearance of uniformity and order, whereas the utilization of a tacky film will most likely result in a film that will dissolve only after sticking to the user's palate for an extended period of time. Furthermore, the modified CMC coatings provide limited weight increase during substrate coating steps [normally between about 0.5 and 8% of the total weight of the target substrate (tablet, beads, microspheroids, etc.), preferably from 1 to 4% weight increase, most preferably from 1.5 to about 3.5%), thereby providing an effective protective barrier to the target substrate as well as a strong, yet lightweight one. The modified CMC materials thus exhibit excellent strength, ease in application (from an aqueous source, primarily, though not necessarily), low friability (if any), and sufficient barrier from exposure to undesirable environmental contaminants, all with a very lightweight addition to a target substrate.

The costs of manufacture have proven difficult to reduce for such previous films, particularly when the amount of film-forming component is relatively low. Solutions of, for instance, hydroxypropylmethyl cellulose (HPMC) including an excess of about 90% or higher by weight of water are typical for such coating materials. Once the solution (syrup) is formed and then applied to a target tablet surface at a substantially uniform thickness, the time required to effectively form the desired film is dependent upon the humidity and temperature of the environment as well as the amount of water required to be evaporated. At such a high level of water, the needed evaporation time is excessive or the amount of heat needed to effectuate such evaporation quickly increases the manufacturing costs to a rather high level. A decrease in water content within the initial solution, although, it may reduce evaporation time ultimately, leads to other problems, most notably the necessity for sufficient mixing to thoroughly disperse such cellulosic materials throughout the solution for proper uniform film production. As such, with too little water present, which results in a too high viscosity, the amount of time and effort required for such needed thorough mixing is inordinately high. In either situation, the cost of manufacture is impacted by the amount of water needed and the ultimate cost for such coating production is ultimately passed on to the consumer.

For the purpose of this invention, the term “coating” is intended to encompass a solid sheet of polymer material that has been applied in a dimensionally stable manner to at least a portion of a target solid form (such as a tablet, a bead, a microsphere, and the like).

Polysaccharides, such as certain cellulosic-based types (carboxymethylcellulose, as one non-limiting example), have been utilized within numerous fields for many years as viscosity modifiers, carriers, anti-redeposition agents, and other like purposes within the paper, oil, food, paint, and detergent industries, to name a few. The benefits of modified cellulosics water-soluble polymers have been provided as well, particularly within U.S. Pat. No. 5,569,483 to Timonen et al., as it pertains to substitution of fat within foodstuffs, and within U.S. Pat. No. and 5,543,162 to Timonen et al., as it pertains to the utilization of such enzymatically modified cellulosics in combination with hydrophilic polymers (such as gelatin) in coacervation methods of forming capsules. There is no discussion within either of these references of the ability of specific modified CMC materials for the purpose of providing excellent film, or other type of coating, particularly those that meet certain molecular weight and thus viscosity requirements.

The present invention relates to an edible tablet (or bead or microsphere) coating composition comprising a safe and effective amount of at least a modified CMC material, optionally, a further amount of another polysaccharide or biogum material, optionally, a safe and effective amount of a plasticizing agent, and optionally, a safe and effective amount of an ingredient, including, as examples, a flavoring agent, a pharmaceutical agent, an oral care additive, an anti-inflammatory agent, an antimicrobial agent, a surfactant, a sweetener, a vitamin, pigments, colorants, and the like. The coatings of this invention may be utilized as protectants for such active ingredients through reliable long-term coating during storage and prior to ingestion by a user. Furthermore, upon introduction within the oral cavity of a user and/or patient, the coating will delay dissolution for a sufficient time to ensure no appreciable taste change due to exposure to the surface of the target tablet (if the tablet is completely coated with the inventive coating) for effective delivery of actives occurring within the user's and/or patient's stomach/gastro-intestinal system.

All percentages and ratios used hereinafter are by weight of total composition, unless otherwise indicated. As used herein, percentage by weight of the film composition means percent by weight of the wet film composition, unless otherwise indicated.

All U.S. patents cited herein are hereby incorporated in their entirety by reference.

The edible tablet coating compositions of the present invention comprise at least one molecular weight-modified CMC material. Although such degradation may be accomplished through any type of well known method, such as acid, radiation, oxidation and heat degradation, preferably the degradation step is provided through enzymatic exposure. Thus, the initial method step is actually providing the CMC material for further use thereof. Such a step may be accomplished similarly to that taught within either of the Timonen et al. patents discussed above. In essence, a CMC having the desired degree of substitution and initial molecular weight is subjected to a preselected amount of cellulase enzyme in order to reduce the overall molecular weight of the CMC material itself to a level proper for coating production. The CMC selected for this step, as alluded to above, must exhibit a proper degree of substitution (i.e., the average amount of carboxymethyl groups per glucose unit) in order to permit the ultimate generation of a tablet coating exhibiting the requisite characteristics of active protection, delayed dissolution, dimensional stability, and low tackiness, at least. For ingestion as tablet coatings, the degree of substitution is preferably, though not necessarily, lower than about 0.95, at most as high as about 1.5. The initial molecular weight may be within a broad range as long as the ultimate molecular weight range meets the requirements that lead to the same type of proper tablet coating generation in terms of the physical characteristics noted above. Thus, an initial molecular weight range, as measured by using GPC analysis of from 80,000 to about 3,000,000 is acceptable. The thus preselected CMC starting material can then be exposed to an amount of cellulase that coincides, in combination with the amount of time of such exposure, pH and temperature with the ultimate degradation of the CMC material into individual strands thereof exhibiting a range of molecular weights from 1,500 to 75,000. If the molecular weight is too low (below 1,500), then the coating will be too friable to properly function. Preferably, though not necessarily, the molecular weight will be between about 20,000 and 50,000 for the modified CMC materials. A lower molecular weight range (i.e., from 1,500 to about 20,000) may be utilized as well, but will preferably, though, again, not necessarily, be compensated for with a higher degree of substitution. After the time of enzyme exposure is completed, the cellulase can then be inactivated through heat exposure, as one example, thereby preventing further degradation of the CMC from occurring. The molecular weight range sought after for the modified CMC materials transfers to a viscosity measurement for the solutions used to ultimately produce the target coatings typically within a range of 150 mPas to 450 mPas. It has been found as well that such viscosity measurements appear to contribute to the overall effectiveness of the ultimately formed coatings in combination with the degree of substitution of the starting CMC materials themselves. Thus, it has been determined that such molecular weight and viscosity properties are critical to the success of the overall invention, at least when the sole coating-forming component of the solution is the modified CMC material.

As noted previously, one surprising result of this invention is that the modified CMC can be utilized as such a sole coating-forming component. Most commercially available films require the utilization of combinations of different polymers to attain desired film properties; however, it has surprisingly been determined that the modified CMC polymers utilized within this invention are sufficient on their own to achieve such results. The ability to form a tablet coating that meets or exceeds the aforementioned physical characteristics as well as can withstand certain salt and relative humidity exposures without appreciably effecting the dimensional stability and usefulness of the ultimate end use product was unexpected. If desired, however, one may include other hydrocolloids, biogums, and/or cellulose ethers to provide increases in salt and/or humidity protection, or to provide viscosity build within pre-applied tablet coating formulations, or to provide gel formation for the same types of formulations, and/or one may include a plasticizer in order to increase film flexibility or provide increases in dimensional stability and other physical characteristics of the subject tablet coatings as well. Such a molecular weight-modified CMC polymer exhibits excellent compatibility with such other possible polymers and thus their optional presence should not be problematic.

The other types of optional polymeric additives that may be utilized within the inventive tablet coatings, again, in addition to the required modified CMC materials, include, without limitation, non-gelling viscosity building additives selected from the group consisting of cellulose ethers, such as methyl cellulose, (non-modified) carboxymethylcellulose, hydroxyethylcellulose, hydroxypropylcellulose, hydroxypropylmethylcellulose, and mixtures thereof; biogums, such as xanthan gum, diutan gum, rhamsan gum and welan gum, gellan gum, and mixtures thereof; and hydrocolloids such as carrageenan, pectin, gum arabic, guar, locust bean gum, gum tragacanth, tara gum, sodium alginate, acacia gum, pullulan, scleroglucan, and mixtures thereof; and any combinations or mixtures thereof such different types of hydrocolloids. Furthermore, other additives that impart gel-forming characteristics to the modified CMC formulations include, without limitation, gel-forming additives selected from the group consisting of of gellan gum (high and low acyl forms), carrageenan (kappa and iota types), xanthan/locust bean gum, sodium alginate, curdlan, MHPC, pectin, and any combinations or mixtures thereof. The optional polymeric additives listed above may be present therein in an amount of from 0.05 to 50% by weight of the entire coating.

In order to obtain a controlled release formulation, it is usually necessary to overcoat the substrate comprising the therapeutically active agent with a sufficient amount of the aqueous solution of the specific carboxymethylcellulose (as defined above) to obtain a weight gain level from about 5 to about 15 percent, although the coating may be lesser or greater depending upon the physical properties of the therapeutically active agent and the desired release rate, the inclusion of plasticizer in the aqueous solution of modified carboxymethylcellulose and the manner of incorporation of the same, for example.

One benefit of utilizing the modified CMC, particularly, whether alone or in combination with these other types of hydrocolloids and/or biogums, is the reduced viscosity exhibited thereby permits greater amounts of the modified CMC to be introduced within the initial film-forming solution (prior to coating) than is customary. As discussed above, this permits a reduction in the amount of water needed for a proper film-forming composition to be produced and drastically reduces the time required for water evaporation. Furthermore, the film-forming solution can be easily and thoroughly mixed under relatively low energy levels such that a properly dispersed solution is accorded the film producer as well. The modified CMC materials are present as long strands, rather than as coiled globules of CMC; thus, the avoidance of detrimental lumps within the film-forming solution is possible at the aforementioned low energy mixing levels. The proper coating-forming solutions thus will comprise from about 10 to about 50% of the modified CMC, from about 50 to about 90% by weight of water, and optionally, from 0 to about 12.5% by weight of a plasticizer.

In addition to the above essential modified CMC coating agents, the coating solution may also comprise other additional film-forming agents other than the hydrocolloids, cellulose ethers, and/or biogums listed above, such as, without limitation, polyvinyl pyrrolidone, polyvinyl alcohol, sodium alginate, polyethylene glycol, polyacrylic acid, methylmethacrylate copolymer, carboxyvinyl polymer, starch, amylose, high amylose starch, hydroxypropylated high amylose starch, dextran, chitin, chitosan, levan, elsinan, collagen, gelatin, zein, gluten, soy protein isolate, whey protein isolate, casein, and mixtures thereof.

It is preferred that the aqueous solution of modified carboxymethylcellulose used in the present invention include an effective amount of a suitable plasticizing agent, as it has been found that the use of a plasticizer will further improve the physical properties of the film. The suitability of a plasticizer depends on its affinity or solvating power for the polymer and its effectiveness at interfering with polymer-polymer attachments as well as the ability of the plasticizer to act as a “swelling agent” for the CMC in the desired solvent (preferably, though not necessarily, water). Such activity imparts the desired flexibility by relieving molecular rigidity and permitting the CMC to form around the desired target substrate. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application. As an aqueous system is preferred, water soluble plasticizers should be utilized in this respect. Thus, preferred plasticizers include polyethyleneglycol, glycerol, and propyleneglycol. Nonaqueous systems may also be utilized. In such an instance, then, the plasticizer should be soluble within such solvents as well.

When the controlled-release coating of the present invention is to be applied to tablets, the tablet core (e.g. the substrate) may comprise the active agent along with any pharmaceutically accepted inert pharmaceutical filler (diluent) material, including but not limited to sucrose, dextrose, lactose, microcrystalline cellulose, xylitol, fructose, sorbitol, dicalcium phosphate, mixtures thereof and the like. Also, an effective amount of any generally accepted pharmaceutical lubricant, including the calcium or magnesium fatty acids may be added to the above-mentioned ingredients of the excipient prior to compression of the tablet core ingredients. Most preferred is magnesium stearate in an amount of about 0.5-3% by weight of the solid dosage form.

The coated tablet formulations of the present invention slowly release the therapeutically active agent, e.g., when ingested and exposed to gastric fluids, and then to intestinal fluids. The controlled release profile of the formulations of the invention can be altered, for example, by varying the amount of overcoating with the aqueous solution of modified carboxymethylcellulose, by varying the amount and type of plasticizer relative to modified carboxymethylcellulose, by the inclusion of additional ingredients or excipients, by altering the method of manufacture, and other techniques.

The coating solutions of the present invention preferably contain, in addition to the film-former, plasticizer, and solvent system (i.e., water), a colorant to provide elegance and product distinction. Color may be added to the aqueous solution of modified carboxymethylcellulose (CMC). For example, color may be added to the modified CMC via the use of alcohol or propylene glycol based color dispersions, milled aluminum lakes and opacifiers such as titanium dioxide, as mere examples. Any suitable method of providing color to the formulations of the present invention may be used.

The plasticized aqueous solutions of modified carboxymethylcellulose may be applied onto the substrate comprising the therapeutically active agent by spraying using any suitable coating equipment known in the art. A sufficient amount of the aqueous solution of modified carboxymethylcellulose to obtain a predetermined controlled release of said therapeutically active agent when said coated substrate is exposed to aqueous solutions, e.g. gastric fluid, is preferably applied, taking into account the physically characteristics of the therapeutically active agent, the manner of incorporation of the plasticizer, etc. After coating with modified CMC, a further overcoat of a film-former, may optionally applied to the target tablet (or microspheres, beads, and the like). This overcoat is provided, if at all, in order to substantially reduce tackiness and possible agglomeration of the tablets (or microspheres, beads, and the like), although the modified CMC should not exhibit such tacky characteristics.

Next, the coated beads are cured in order to obtain a stabilized release rate of the therapeutically active agent. Curing is traditionally carried out, if at all, via a forced-air oven at 60° C. for anywhere from 2-24 hours or in-line.

The compositions of the present invention may also comprise a safe and effective amount of an additive selected from the group consisting of a flavoring agent, an antimicrobial agent, a surfactant, a sweetener, and any combinations thereof.

Suitable flavoring agents include any well known food flavoring (of which there are a vast variety to choose from) including, without limitation, examples such as oil of wintergreen, oil of peppermint, oil of spearmint, clove bud oil, menthol, eucalyptol, lemon, orange, cinnamon, vanillin, and the like, and mixtures thereof. In another embodiment, in order to stabilize the flavor, the compositions may optionally comprise a vegetable oil.

Antimicrobial agents (preservatives) may also by optionally present in the present compositions. Such agents may include, but are not limited to alcohols, propylparaben, and methylparaben. Suitable surfactants are those which are reasonably stable and include nonionic, anionic, amphoteric, cationic, zwitterionic, and mixtures thereof.

The present compositions may optionally comprise sweetening agents including sucralose, sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and mixtures thereof.

### Preferred Embodiments of the Invention

The coating compositions utilized in accordance with the invention are formed by processes conventional in the tablet coating art. Generally, the separate components of the coating solutions are blended in a mixing tank until a homogeneous mixture is achieved. Thereafter, the coating solutions can be applied onto an appropriate tablet substrate by spraying, fluid bed drying and other coating techniques known in the tablet coating art, to an acceptable thickness. The coated tablets are then dried (cured), e.g. in a forced-air oven or in-line. The temperature of the drying air and length of drying time depend on the nature of the solvent utilized as is recognized in the art. Most of the coatings contemplated herein, however, are dried at a temperature between about 25° C. (i.e., ambient temperature) and 140° C. (with a lower temperature preferred to reduce costs), for a duration of about 20 minutes to about 60 minutes, in another embodiment from about 30 to about 40 minutes. Drying of these coated tablets should be carried out in a way that the actives included therein are not deleteriously affected by exposure to the necessary level of heat. When dried properly, the coatings will be non-tacky and will have a final water activity of 0.5 (±0.25) so that they do not either take up or lose significant amount of water when exposed to normal ambient conditions. The moisture content will vary depending upon the composition of the coating, its water activity rather than water content that is the parameter to be controlled. Coatings with a low water content may be dried in as little as 30 minutes at 40° C. The optimal temperature of the film during drying is usually lower than 65 C°. Higher temperatures can be used, especially if the film is dried simultaneously from the top and bottom.

Extrusion is also a possible method of coating solution manufacture. The mechanical particulars of the extrusion process, e.g. the particular equipment utilized, the extruding force, the shape and temperature of the orifice are considered to be within the skill of the art and can be varied in a known manner to achieve the physical characteristics of the films described herein.

The coatings herein are generally between about 0.1 and about 10 mils (about 0.025 mm to about 0.25 mm), in another embodiment from about 0.2 to about adjust this one 2.5 mils (about 0.03 mm to about 0.100 mm) thick, and will effectively provide a uniform coating over a target tablet upon application thereon. A uniform application on the surface of a target tablet should be achieved therewith. In particular, the coatings should be applied at an overall weight increase of at most 8% of the weight of the target substrate, and at least 0.5%. Outside of this range would create too great a weight increase without any increase in protective characteristics or an insufficient level of protection to the target substrate. Preferably, the amount of coating is added at a weight increase of from about 1 to 5% by weight of the target substrate, most preferably from 1.5 to 3.5%.

The processes followed for production of the inventive modified CMC materials and tablet coatings made therefrom are delineated below.

### EXAMPLES

### 1) Modified CMC Production

Samples of different CMC materials were modified to different levels of molecular weights in order to provide materials for ultimate film coating production. In each instance, the basic degradation method was preferably performed enzymatically and followed the basic steps of: Tap water was charged to a barrel that was placed in a water bath of 50° C. From a food grade cellulase (Econase CE from AB enzymes) from Trichoderma reesei, 0.1-1% (weight percent on dry CMC basis) was added to the water (exhibiting a pH of 5.8 as adjusted by a 21% phosphoric acid solution). While stirring thoroughly CMC from CPKelco (the different types are noted within Table 1, below) was slowly added over a period of an hour to a concentration of 20% in water. The pH was then adjusted again to 5.8 using the same phosphoric acid solution. The reaction was performed at 50° C. while stirring for 16 hours and was eventually stopped by inactivating the enzyme in an autoclave at 121° C. for one hour. The resultant modified CMC solutions were then dried by either freeze-drying or spray drying.

TABLE 1
Characteristics of Modified CMCs

| | CMC starting | | Mod CMC | |
|---|---|---|---|---|
| | material | Mod CMC | Mol. Weight | Enzyme |
| Ex. | Tradename | Deg of Subst. | (Dalton) | Amt. (% w/w) |
| 1 | CEKOL ® 30000A | 0.91 | 19500 | 0.1% |
| 2 | CEKOL ® 2000S | 1.26 | 22500 | 1.0% |
| 3 | CEKOL ® 50000 | 0.60 | 3500 | 1.0% |

### 2) Tablet Manufacturing

Curved placebo tablets with break line were made using a Korsch EK0 tablet press and the following formulation:
48% w/w lactose
48% w/w dicalcium phosphate
3% NYMCEL® ZSX (from CPKelco Oy)
1% Magnesium stearate

Physical properties of the tablets:
The hardness of the tablets: 10.4 Kp
tablet weight: 0.5 g
tablet thickness (at thickest point): 5.0 mm
tablet diameter: 10.1 mm

### 3) Film Production

TABLE 2
Cellulose ethers used for film production

| Ex. | Tradename |
|---|---|
| 1 | CEKOL ® 30 |
| 2 | modified CMC, Ex. 1 from Table 1 |
| 3 | hydroxypropylmethyl cellulose (METHOCEL ® E5) |
| 4 | modified CMC, Ex. 2 from Table 1 |
| 5 | modified CMC, Ex. 3 from Table 1 |

The materials from Table 2, above, were utilized to form films in accordance with the following method: The material was weighed out and dissolved into tap water. After the material was dissolved completely, plasticizer was weighed out and added to the dissolved modified CMC solution. Air bubbles within the resultant solution were removed by centrifugation or by vacuum. That solution was then cast using a draw-down bar on a plastic sheet into thin even layers. The layers were then dried at room temperature to form films exhibiting final thicknesses of between 20 and 500 μm.

Table 3, below, thus indicates the different films produced with the plasticizer (i.e., glycerol) to modified CMC ratio. Note that the remainder of the solution utilized to form the films was tap water (thus, if 40% was CMC, and the plasticizer:CMC ratio is 1:10, then 4% of the solution was plasticizer, and 56% was then tap water, for example). Also, if no plasticizer was added, the term “None” is used and thus the remainder of the film-producing solution was tap water alone.

TABLE 3
Films Produced from Cellulose Ether Materials

| | Cellulose ether from Table 2 | |
|---|---|---|
| Film Ex. | (concentration in %) | Plasticizer:CMC Ratio |
| 1 | 1 (12) | none |
| 2 | 1 (12) | 1:10 glycerol |
| 3 | 1 (12) | 1:10 polypropylene glycol |
| 4 | 1 (12) | 1:10 PEG 400 |
| 5 | 1 (12) | 1:10 PEG 2000 |
| 6 | 1 (12) | 1:10 PEG 6000 |
| 7 | 2 (40) | none |
| 8 | 2 (40) | 1:10 glycerol |
| 9 | 2 (40) | 1:10 polypropylene glycol |
| 10 | 2 (40) | 1:10 PEG 400 |
| 11 | 2 (40) | 1:10 PEG 2000 |
| 12 | 2 (40) | 1:10 PEG 6000 |
| 13 | 3 (20) | None |
| 14 | 3 (20) | 1:10 glycerol |
| 15 | 3 (20) | 1:10 polypropylene glycol |
| 16 | 3 (20) | 1:10 PEG 400 |
| 17 | 3 (20) | 1:10 PEG 2000 |
| 18 | 3 (20) | 1:10 PEG 6000 |

These resultant films were then analyzed for various physical characteristics as noted below. Note that not all of the films produced within the Table 3 above were analyzed using each method below.

### 4) Analysis of the Films

Mechanical Properties—Certain properties, such as tensile strength, toughness, and elastic modulus were measured for resultant films as well to indicate the viability of such films as potential commercial products. Such measurements were taken through standard techniques. A texture analyzer (TA-XT plus) from Stable Micro Systems equipped with tensile grips was used to determine the mechanical properties of the films at 40% RH. The average of 10 measurements was calculated and shown in Table 4.

TABLE 4
Mechanical Properties of Pre-Coating Films

| Film ex. | | | |
|---|---|---|---|
| from | Tensile Strength | Toughness | E-modulus |
| table 3 | (N/mm²) | (N/mm² * %) | (N/mm²/%) |
| 1 | 59 | 255 | 24 |
| 2 | 56 | 301 | 22 |
| 3 | 49 | 125 | 22 |
| 4 | 50 | 167 | 24 |
| 6 | 51 | 161 | 26 |
| 7 | 60 | 98 | 26 |
| 8 | 45 | 54 | 22 |
| 9 | 43 | 65 | 21 |
| 10 | 46 | 126 | 16 |
| 11 | 30 | 33 | 17 |
| 12 | 33 | 39 | 18 |
| 13 | 47 | 436 | 13 |
| 14 | 29 | 265 | 11 |
| 15 | 30 | 192 | 12 |
| 16 | 30 | 194 | 10 |
| 17 | 26 | 375 | 13 |
| 18 | 35 | 410 | 10 |

### 5) Coating Preparation and Viscosity Measurements.

The cellulose ether was weighed out and dissolved into tap water. After the material was dissolved completely, plasticizer was weighed out and added to the dissolved cellulose ether solution. A few drops color solution (1% erythrosine) were added in order to review the coating uniformity. Air bubbles within the resultant solution were removed by centrifugation or by vacuum. The solutions were used for film coating the tablets and viscosity measurements.

TABLE 5
Viscosity of the Coating Solutions

| | Cellulose ether | | |
|---|---|---|---|
| | from Table 2 | | |
| | (concentration | | Brookfield |
| Coating | in | | Viscosity @ |
| solution | %) | Plasticizer:CMC Ratio | 25° C. LV 2/60 RPM |
| 1 | 1 (3) | None | 90 |
| 2 | 1 (3) | 1:10 glycerol | 93 |
| 3 | 1 (3) | 1:10 polypropylene | 91 |
| | | glycol | |
| 4 | 1 (3) | 1:10 PEG 400 | 90 |
| 5 | 2 (15) | None | 69 |
| 6 | 2 (15) | 1:10 glycerol | 74 |
| 7 | 2 (15) | 1:10 polypropylene | 72 |
| | | glycol | |
| 8 | 2 (15) | 1:10 PEG 400 | 74 |
| 9 | 2 (16) | 1:10 PEG 400 | 123 |
| 10 | 2 (18) | 1:10 PEG 400 | 147 |
| 11 | 2 (20) | None | 201 |
| 12 | 2 (20) | 1:10 glycerol | 231 |
| 13 | 2 (20) | 1:10 polypropylene | 228 |
| | | glycol | |
| 14 | 2 (20) | 1:10 PEG 400 | 243 |
| 15 | 3 (8) | None | 190 |
| 16 | 3 (8) | 1:10 glycerol | 195 |
| 17 | 3 (8) | 1:10 polypropylene | 207 |
| | | glycol | |
| 18 | 3 (8) | 1:10 PEG 400 | 204 |
| 19 | 4 (20) | 1:10 PEG 400 | 91 |
| 20 | 5 (27) | 1:10 PEG 400 | 207 |

### 6) Coating Process

Tablets were coated using a central motor unit (AR 402) from Erweka equipped with a DKS 15 liter coating pan.

The angle of the pan was adjusted to get an optimum result from the coating process. The coating pan was also equipped with hot air blower (Carmen Advanced 2000) to accelerate the drying process.

An air spray gun from Max Air equipped with a 14 mm nozzle was used at 3 bar air pressure.

### 7) Tablet Properties

i. Weight Gain of the Tablets

The tablets were weighed before and after the coating process.

TABLE 6
Weight Gain Due to the Coating Process

| | Coating solution | |
|---|---|---|
| Coating trial | Ex. from Table 5 | Weight gain (%) |
| 1 | 18 | 0.8 |
| 2 | 18 | 1.4 |
| 3 | 18 | 1.6 |
| 4 | 10 | 5.2 |
| 5 | 10 | 2.4 |
| 6 | 10 | 7.2 |
| 7 | 4 | 1.0 |
| 8 | 4 | 1.2 |
| 9 | 19 | 5.2 |
| 10 | 19 | 4.4 |
| 11 | 19 | 6.6 |
| 12 | 19 | 3.4 |
| 13 | 20 | 2.0 |
| 14 | 20 | 5.2 |
| 15 | 20 | 4.2 |

ii. Tablet Thickness

The increase in tablet size due to the coating process was measured using a digital micrometer, MITUTOYO® M1-15QM. The average of 6 tablets was calculated for each example.

TABLE 7
Coating Thickness

| Coating trial | Coating solution | Coating thickness |
|---|---|---|
| Ex from Table 6 | Ex from Table 5 | (μm) |
| 1 | 18 | 26.5 |
| 2 | 18 | 31.5 |
| 3 | 18 | 32 |
| 4 | 10 | 105.5 |
| 5 | 10 | 48.5 |
| 6 | 10 | 135 |
| 7 | 4 | 37 |
| 8 | 4 | 44.4 |
| 9 | 19 | 74.4 |
| 10 | 19 | 72.1 |
| 11 | 19 | 104 |
| 12 | 19 | 43.8 |
| 13 | 20 | 26.3 |
| 14 | 20 | 69.1 |
| 15 | 20 | 58 |

iii. Tablet Hardness/Crushing Strength

Tablet hardness also known as the crushing strength, was determined using the tablet hardness tester SCHLEUNIGER® 5Y. The average of 10 tablets was calculated.

TABLE 8
Tablet Hardness

| Coating trial | Coating solution | |
|---|---|---|
| Ex from Table 6 | Ex from Table 5 | Hardness (kP) |
| Uncoated tablets | — | 10.4 |
| 1 | 18 | 12.0 |
| 2 | 18 | 12.7 |
| 3 | 18 | 13.4 |
| 4 | 10 | 24.5 |
| 5 | 10 | 15.0 |
| 6 | 10 | 32.6 |
| 7 | 4 | 16.6 |
| 8 | 4 | 17.8 |
| 11 | 19 | 25.8 |
| 15 | 20 | 14.1 |

iv. Disintegration Time

The disintegration time of tablets is the time that is needed for a tablet to completely disintegrate when contacting a solvent. The Erweka® ZT71 disintegration tester was used to automatically determine the disintegration time of 6 individual tablets of one batch at the same time as described in the pharmaceutical technical procedure in the European pharmacopoeia “disintegration of tablets and capsules”. The solvent used is water and the temperature was 37° C.

TABLE 9
Disintegration Time

| Coating trial | Coating solution | Disintegration time |
|---|---|---|
| Ex from Table 6 | Ex from Table 5 | (s) |
| Uncoated tablets | — | 40 |
| 1 | 18 | 50 |
| 2 | 18 | 62 |
| 3 | 18 | 62 |
| 4 | 10 | 85 |
| 5 | 10 | 68 |
| 6 | 10 | 96 |
| 7 | 4 | 68 |
| 8 | 4 | 70 |
| 11 | 19 | 82 |
| 15 | 20 | 59 |

v. Friability

The friability of tablets is the weight loss that appears after 100 times tumbling at 25 RPM. Also the friability test fails if obviously cracked, cleaved or broken tablets are present after tumbling. The Erweka TAR-100 was used to measure the friability of the tablets as described in the pharmaceutical technical procedure in the European pharmacopoeia.

The weight loss of the uncoated tablets is 0.22%. No weight loss of the coated tablets was observed.

Thus, surprisingly, the tablets coated with the modified CMC exhibited excellent properties in all respects, all with limited levels of weight increase to the target substrates, and quick film-production times due to low evaporation requirements from the aqueous solutions made therefrom.

While the invention will be described and disclosed in connection with certain preferred embodiments and practices, it is in no way intended to limit the invention to those specific embodiments, rather it is intended to cover equivalent structures structural equivalents and all alternative embodiments and modifications as may be defined by the scope of the appended claims and equivalence thereto.

What is claimed:

1. A substrate selected from a tablet, a bead, and a microsphere, said substrate coated with a composition comprising modified CMC materials exhibiting a molecular weight range of from 1500 to 75000 and a degree of substitution of less than about 1.5; wherein said composition optionally comprises at least one polymeric additive other than said modified CMC materials.

2. A method of producing such a tablet coating comprising the steps of a) providing a CMC material exhibiting a molecular weight range of from 80000 to 3000000 and degree of substitution of less than about 1.5; b) degrading said CMC materials by exposing said materials to an enzyme in an amount and for a period of time sufficient to reduce the molecular weight range of said CMC materials to a range of from 1500 to 75000; c) inactivating said enzyme; d) producing a solution of the resultant modified CMC materials of step “b” with at most 70% by weight of water and optionally including at most 12.5% of a plasticizer; e) providing a solid tablet formulation; and f) applying said resultant modified CMC materials of step “d” to the at least a portion of the surface of said tablet formulation of step “e”, thereby allowing said water therein to evaporate therefrom.
